(19) United States
(12) Patent Application Publication
(10) Pub. No.: US 2026/0263694 A1
(43) Pub. Date: Sep. 10, 2026

(54) SYSTEM AND METHOD FOR INTELLIGENTLY CONTROLLING MEDICAL DEVICES

(71) Applicant: CareFusion 303, Inc., San Diego, CA (US)

(72) Inventors: Daniel M. ABAL, San Diego, CA (US); Brendan John BURGESS, Poway, CA (US); Ramkumar SUBRAMANIAN, San Diego, CA (US); Brandon FRIDLEY, San Diego, CA (US)

(21) Appl. No.: 18/723,307

(22) PCT Filed: Dec. 21, 2022
(86) PCT No.: PCT/US2022/053722

§ 371 Date: Jun. 21, 2024

Related U.S. Application Data
(60) Provisional application No. 63/293,620, filed on Dec. 23, 2021.

(51) Int. Cl. A61M 5/168; G16H 20/17; G16H 40/63

(52) U.S. Cl. CPC A61M 5/16804; A61M 5/16877; G16H 20/17; G16H 40/63; A61M 2205/14; A61M 2205/18; A61M 2205/3303; A61M 2205/502

(57) ABSTRACT

An external infusion control device operably connects to an infusion device and one or more sensors, and is configured to control the infusion device based on sensor data provided by the sensors and user input provided at the control device. The control device provides a user interface to a display and, on receiving a therapy selected by a user, confirms the sensors for the therapy are operably connected to the control device and that the control device is operably connected to an infusion device that may be controlled by the sensor data. One or more algorithms are obtained, for example by way of being downloaded from a server, to control the infusion device based on the sensor data, and performance of the selected therapy is facilitated by the control device based on sensor data received from the one or more sensor devices.

### TECHNICAL FIELD

The present disclosure is generally related to a control device configured to facilitate operation of a medical device, and which is configured to communicate with a remote device.

### BACKGROUND

Patient care units may include modular infusion platforms that are expandable with multiple medication delivery modules to handle more than one type of medication delivery to a patient. Different patients require different levels of treatment, and different parameters for different devices. Each individual infusion device may operate differently, and include a different type of user interface, resulting in a wide variety of display types and parameter variations that may confuse or distract clinicians during infusion and other medication delivery procedures. During surgery and under conditions where direct access to the infusion device or devices may be difficult, precise control and coordination of the devices may be difficult. Failure to maintain control over the devices increases the health risk to the patients of a healthcare facility. For example, under manual control the amount of time a patient is provided medication at a level that is within a desirable range can fluctuate based on, for example, the attentiveness of the clinician. The amount of time a patient spends outside the desired range can have an impact on the procedure, patient, recovery, and resources used for administering the therapy. Furthermore, some therapies require repetitive work steps that can be prone to error or complacency, which may lead to an adverse event.

### SUMMARY

According to various aspects, the subject technology provides a system and method for intelligently controlling medical devices, and more particularly infusion devices. The intelligent control module (ICM) of the subject technology provides direct control over connected medical devices to assist the clinician in providing a more centralized control and management of the devices. The ICM provides a modular interface system by which various sensors used in a medical environment may be connected in a generic way to facilitate control over the connected medical devices. For example, a heart rate monitor, oxygen sensor, and an IV flow rate monitor may all be connected to the ICM to facilitate, in addition to input by the clinician, centralized control of one or more infusion devices. The ICM may further connect to server and cloud-based systems for further data input, data coordination, and reporting.

According to various aspects, an infusion control device includes: a non-transitory machine-readable memory; and a processor operably connected to a display and the memory, the processor configured to: provide a user interface to the display; prompt, via the user interface, a user to select a therapy; receive a selected therapy from the user interface; confirm that one or more sensor devices required for the selected therapy are operably connected to the infusion control device; confirm that an infusion device is operably connected to the infusion control device and that the infusion device is controllable based on information derivable from the sensor devices; obtain a first algorithm configured to operate the infusion device based on real-time data received from the one or more sensor devices; and facilitate performance of the selected therapy based on sensor data received from the one or more sensor devices. Other aspects include corresponding systems, methods, and computer program products for implementation of the corresponding device and its features.

A method includes receiving an indication that a control device is operably connected to one or more infusion devices, the control device being physically distinct from the one or more infusion devices; presenting, for the control device, a user interface configured to provide for selection one or more predetermined therapies associated with the one or more infusion devices and configured to display one or more graphical data modules based on the selection; receiving, at the control device, a selection of a first therapy of the one or more predetermined therapies provided by the user interface; and based on receiving the selection of the first therapy, at the control device: determining that one or more sensor devices for providing data associated with the selected first therapy are operably connected to the control device; downloading to the control device, from a server based on determining that the one or more sensor devices are operably connected to the control device, a first algorithm configured to operate the one or more infusion devices based on real-time data received from the one or more sensor devices; generating a default organization of one or more respective graphical data modules within the user interface based on the one or more sensor devices and the downloaded first algorithm, wherein at least one of the one or more respective graphical data modules is configured to receive a configuration parameter associated with operation of the one or more infusion devices; prompting for the configuration parameter; receiving, via the at least one respective graphical data module, the configuration parameter; and controlling the one or more infusion devices based on the real-time data received from the one or more sensor devices, the downloaded first algorithm, and the received configuration parameter. Other aspects include corresponding systems, apparatus, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts an example of an institutional patient care system of a healthcare organization, according to aspects of the subject technology.

FIG. 1B illustrates an example patient care unit, including a control unit and connected medication delivery modules, according to various aspects of the subject technology.

FIG. 2A is a conceptual diagram illustrating an example infusion control system integrating an example decision module with one or more medical systems and one or more sensing devices, according to aspects of the subject technology.

FIG. 2B depicts an example modular user interface configured to dynamically connect to and receive data from different sensors to control different infusion therapies, according to aspects of the subject technology.

FIG. 3 depicts an example process for intelligently controlling an infusion, according to aspects of the subject technology.

FIG. 4 is a conceptual diagram illustrating an example electronic system for intelligently controlling an infusion, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology provides an intelligent control module (ICM), including a unit that provides processing for control algorithms, and connectivity to enable closed and semi-closed-loop control capabilities over one or medical devices at a point of use. In some implementations, the ICM provides an external interface between an IV infusion pump and one or more different physiological sensors, and provide input parameters that are to be used for controlling the titration of IV infusions of medications to a patient. In this regard, the ICM can incorporate control software (including, e.g., one or more algorithms) that can be tailored to specific or general medical treatments.

A closed-loop control system generally refers to a system that does not rely on external manual inputs to deliver a therapy. Once configured, the closed-loop system can autonomously provide a therapy, receive feedback from one or more sensors, and, based on the feedback, automatically adjust the therapy as needed. A semi-closed-loop control system is similar to a closed-loop control system except that in some circumstances, the adjustment to the therapy may depend on an external input. In some implementations, a semi-closed-loop control system may be referred to as a decision support system.

According to various implementations, the ICM facilitates the control software to be separate from the embedded firmware of both the pumps, and from sensors, which can facilitate a scalable and rapidly configured system to provide closed-loop control of medical treatments. Moreover, the ICM can be configured to operate with a multitude of sensors by way of electrical connectors and/or wireless communication. The ICM may include one or more microprocessors and algorithms to provide signal conditioning and/or conversion of the sensor signals to the appropriate physiological parameters for the connected medical device. The parameters may then be used in control algorithms to provide control to, for example, an infusion pump to deliver the necessary medications or fluids for a desired clinical outcome. As used herein, “connecting” devices or “operably connecting” devices may include establishing a physical (e.g., wired) or virtual (e.g., wireless) connection between the devices.

Having the ICM separate unit from the medical device provides several advantages. For example, the advancement of sensors may be much more rapid than the development of infusion pump systems, and thus the control system may accommodate these changes more quickly. It may be desirable to update control algorithms to address changes in treatment methods, available medications, and patient physiology. For this reason, it may be desirable to have the algorithm reside in a component different than the pump system, in order to accommodate more frequent changes. Moreover, machine learning and artificial intelligence may account for patient variations related to physiological parameters such as age, genetics, health history, and other characteristic and environmental factors. Systems incorporating such capabilities may involve large databases and complex programs requiring powerful microprocessors and data storage capabilities to perform the timely and accurate computation needed. These systems are generally not capable of running on the systems currently available with the IV pumps alone.

Additionally, the ICM of the subject technology is adaptable to a variety of pump systems and sensor inputs. The ICM may further be configured to add wireless, Bluetooth and LAN connections to pump systems that do not currently have it available. Adding such communications to the pump system may enable other capabilities such as remote monitoring and control of the infusion pumps, and the access to patient EMR. Accordingly, by integrating the electrical and processing components separate from the pump, the subject technology facilitates integration of additional capabilities without needing to modify the pump's housing and electronics. Separating the physiological sensing and control systems from the infusion pump system may further provide for a more streamlined regulatory approval process.

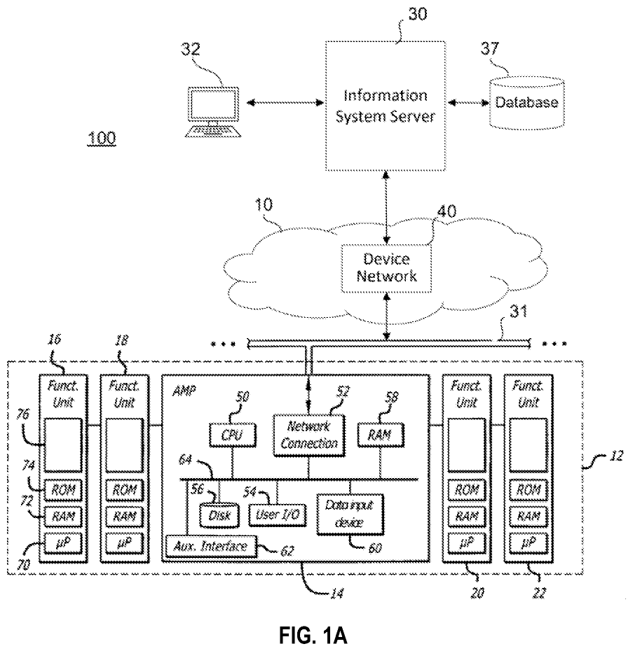

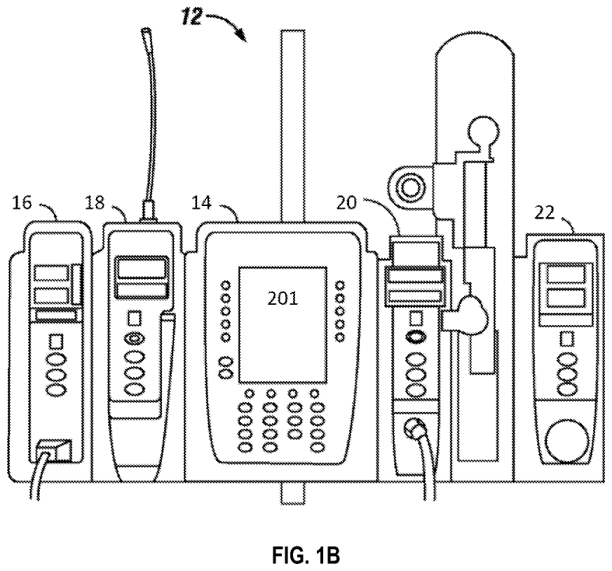

FIG. 1A depicts an example of an institutional patient care system 100 of a healthcare organization, according to aspects of the subject technology. In FIG. 1A, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1A optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care devices 12 (and other devices) communicate in accordance with normal operations.

Additionally, institutional patient care system 100 may incorporate a separate information system server 30, the function of which will be described in more detail below. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistants, mobile devices such as laptops, tablet computers, augmented reality devices, or smartphones, configured with software for communications with information system server 30 via network 10.

Patient care device 12 comprises a system for providing patient care, such as that described in U.S. Pat. No. 5,713,856 to Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 16, 18, 20, 22. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally, or in the alternative, data input device 60 can be any device for entering coded data into a computer, such as a device(s) for reading a magnetic strip, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1A to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 1A, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor or an intracranial pressure monitor or the like. Functional module 18, 20 and/or 22 may be a printer, scanner, bar code reader or any other peripheral input, output or input/output device.

Each functional module 16, 18, 20, 22 communicates directly or indirectly with interface unit 14, with interface unit 14 providing overall monitoring and control of device 12. Functional modules 16, 18, 20, 22 may be connected physically and electronically in serial fashion to one or both ends of interface unit 14 as shown in FIG. 1A, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1A, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 16.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction, or a combination of both automated and manual interaction. Automated interaction may be continuous or intermittent and may occur through direct network connection 54 (as shown in FIG. 1A), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication

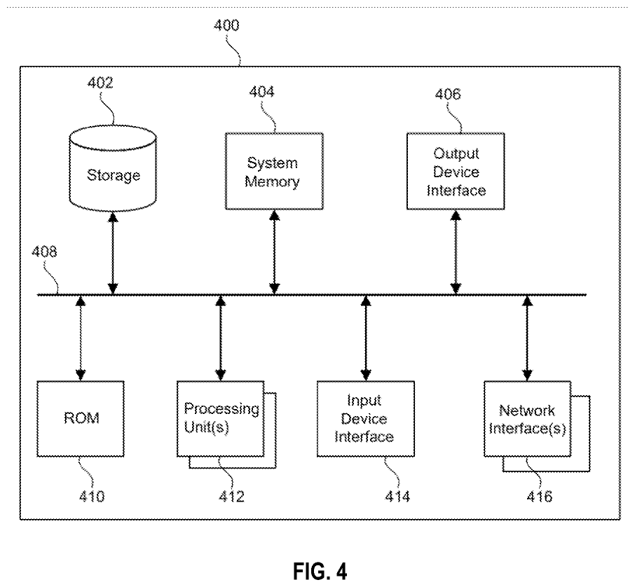

FIG. 1B illustrates an example PCU 12, including a control module 14 together with connected medication delivery modules 16, 18, 20, 22, according to various aspects of the subject technology. In some embodiments, medication delivery modules 16, 18, 20, 22 include plug-in ports for expansion. Accordingly, a new medication delivery module may be attached to PCU 12 by coupling a connector through the plug-in ports, which may include electrical terminals so that the added medication delivery module 16, 18, 20, 22 may transmit and receive information to and from a control module 14. In some embodiments, the added medication delivery module 16, 18, 20, 22 may also receive power from control module 14 through a plug-in port. Control module 14 may include a main display 201, a memory and a processor (see FIG. 4), and may be configured to display operational parameters and medication delivery status, and further information associated with each of medication delivery modules 16, 18, 20, 22. According to various implementations, module displays may also display physiological data (e.g., vital signs) associated with a patient.

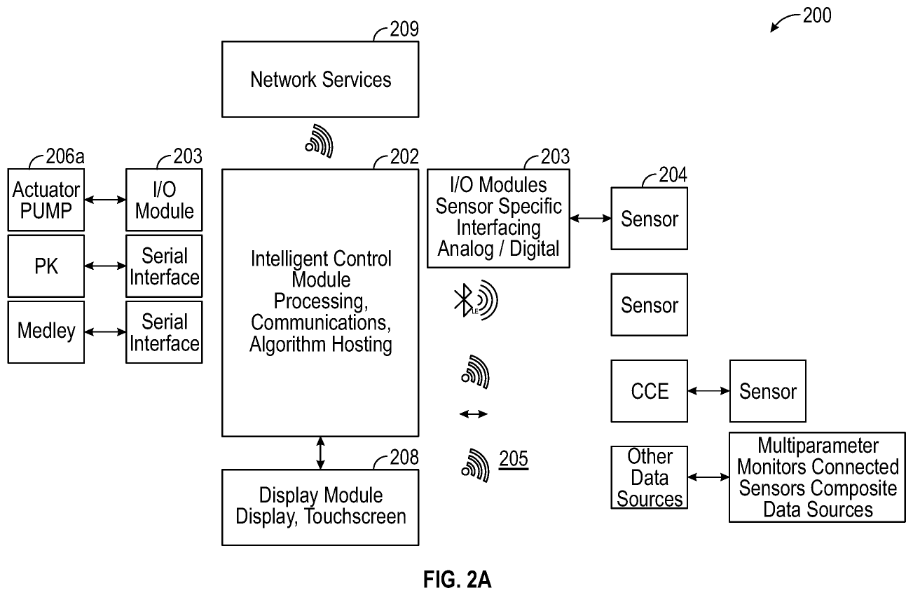

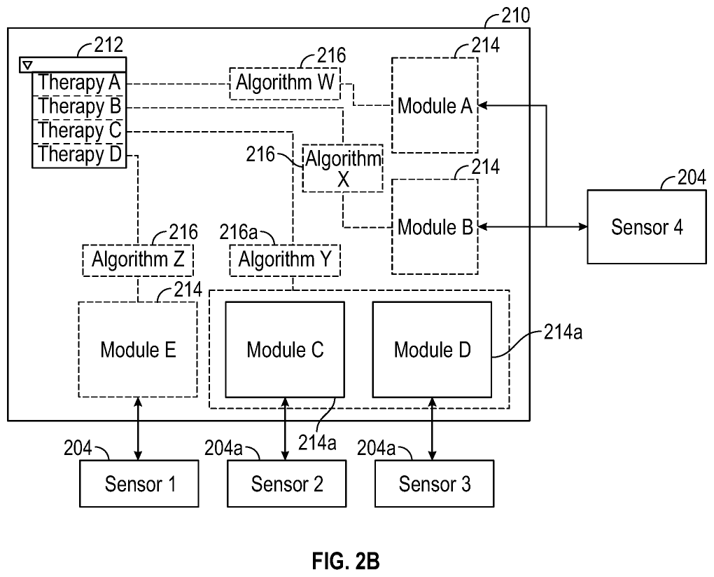

Main display 201 is configured to display one or more user interfaces 210 (see FIG. 2B) for the display of operational parameters or other data associated with a module 16, 18, 20, 22, and/or physiological parameters associated with the patient. Main display 201 may include multiple user interfaces 210, with each individual user interface graphically displaying information for a respective one of medication modules, including information also displayed on a corresponding module displays. In some embodiments, control module 14 includes a communications module (including, e.g., an antenna), configured to communicate wirelessly with a controller, or with a network.

With reference to FIGS. 1A and 1B, when a medication delivery module 16, 18, 20, 22 initiates an infusion of a medication to the patient, the control module 14 is configured to create and manage an infusion session within a memory of the control module (or related module). For the purpose of this disclosure, the infusion session includes state information of the PCU 12, its control module 14, and/or its associated modules, which is recorded and saved to memory during a particular period of time. The state information includes, but is not limited to, records of parameter values utilized by the PCU, its control module, and/or its associated modules during the period of time, and/or records physiological data collected during the period of time. During the infusion, physiological data associated with the patient is recorded within the session, operating parameter values, and any modifications to the operating parameters of the PCU, its control module, and/or modules are also recorded in the session.

If not already logged into the PCU 12, the clinician may scan his or her badge proximate to a sensor (e.g., 54, 60) on the PCU 12, and the PCU may attempt to authenticate the clinician by sending the clinician's scanned identification to server 30. The clinician's badge may incorporate a radio frequency identification device (RFID), which is read by a scanner integrated with the PCU, or a portable scanner associated with the PCU. The clinician may scan his or her badge at the control module 14 to identify and authorize the clinician to initiate the administration of a medication. Once the clinician is associated with the PCU and/or module(s), the clinician's identification is associated with the session. The same is applicable with a patient. The clinician may scan the patient's wristband with a portable scanner, or using the sensor on the PCU 14 (or its control module) to associate the patient with the PCU and/or module(s) (and a session).

The control unit 14 of PCU 12 is configured to generate a graphical representation of the infusion session, and display (e.g., in display 201) the graphical representation, including a graphical visualization of all parameters of the infusion during the session and any modifications any modifications to the parameters, together with physiological data obtained during the session. The graphical representation may include pseudo identifiers for unknown data until such data is substituted with known identifiers. At that time, the graphical representation is displayed with the known patient identifiers.

FIG. 2A is a conceptual diagram illustrating an example infusion control system 200 integrating an example decision module with one or more medical systems and one or more sensing devices, according to aspects of the subject technology. In the depicted example, an ICM 202 includes multiple configurable hardware interfaces 203 that receives input from various sensors and data sources 204. The ICM is simultaneously connected to, via the interfaces 203, one or more medical devices 206 including, for example, an infusion pump 206a such as an actuator pump, syringe pump (e.g., BD Alaris™ PK syringe pump), a large volume infusion pump (e.g., BD Alaris™ Plus infusion pump), or a modular infusion pump (e.g., BD Alaris™ infusion system or BD Alaris™ Medley infusion pump). The ICM may incorporate an algorithm that remotely controls an IV infusion of medication or fluids by the infusion pump. As will be described further, the ICM 202 may be configured to dynamically load one or more algorithms to control an infusion in different ways. For example, an algorithm may control a Pharma Kinetic (PK) infusion, Titration Controlled Infusion (TCI) infusion, Proportional Integrative Derivative (PID) infusion, or other proprietary control infusion.

Depending on the sensor I/O requirements or signal processing requirements, the ICM 202 may be configured with adapters 203 that support the connection of varied sensors 204 and/or medical devices 206. Sensors may require analog or digital I/O with specific power supply requirements. A modular I/O adapter 203 can interface to standard digital I/O available in the decision module, i.e., USB, UART, data bus. The I/O module in turn interfaces to the external sensor needs, e.g., analog, optical, custom wireless such as UWB, Zigbee, Bluetooth low energy. The I/O module may contain additional processing i.e., microcontroller, or digital signal processor hardware. The I/O module may be designed to support multiple sensors or support a single vendor supplied sensor.

According to various implementations, embedded firmware incorporated in each I/O module 203 and/or software drivers supporting the I/O module types form an extensible “plug and play” capability. I/O modules 203 may be added to the ICM 202 as needed. If certain I/O functionality is deemed common or needed by default, then a predetermined adapter capability (e.g., BLE, standard serial interface, etc.) may be included in the decision module by default.

In some implementations, sensor input (e.g., for closed-loop control) may be acquired over a network interface 205. In one example, a variety of sources that connect one or more clinically relevant sources of input to the ICM 202 may provide data to support control decisions (including, e.g., controlling the closed-loop use case). One example may include sensor input from a patient care system such as the CareFusion Care Coordination Engine (CCE). The CCE may receive data from an electronic medical record or directly from a sensor or other device and transmit the data via the network interface 205. Another input may include a multi-parameter monitor.

IV infusion devices 12 or other delivery devices may be directly controlled by the ICM 202 using serial, wired or wireless network connectivity (Ethernet or WIFI), Other wireless connectivity such as BLE. Where specialized connectivity might be required then a predetermined I/O module 203 corresponding to the type of device may be connected. Accordingly, external devices that may be connected to the ICM 202 may include, for example, a badge RFID reader (e.g., for tasks such as NFC tap to associate, patients, sensors, pumps, clinician login), a bio identification device (e.g., a fingerprint or retina scanner), or a backup battery for power loss or ambulatory usage.

The ICM 202 further includes a display module 208 configured to provide a user interface for display of information pertaining to patient physiological status, as well as system control status. In some implementations, display module 208 includes circuitry within the ICM 202 housing that provides display information to an external display device. Being able to connect to another display provides modular scalability. For example, if the use case requires a rich user interface with clinician displays including data and graphs, a larger high-resolution display could be used. If the use case requires a display with minimal information and UI to support configuration, then a smaller, space saving and lower cost locally connected display could be used.

The ICM may be connected to a local display by cable or directly attached to form a combined module pair. The ICM 202 may also be configured with minimal or no local display (e.g., “headless”) capabilities, and configured to wirelessly connect to a mobile device such as a smartphone or tablet (e.g., via BLUETOOTH) and to display information via a mobile application operating on the remote device. Display information may also be provided to an external clinician display or portal for display with other information specific to the portal. In some implementations, the ICM may include an integrated display device. In some implementations, the ICM 202 may share a display with one or more medical devices 206. For example, both an infusion pump 206a and the ICM 202 may share a single external display for the presentation of information and/or control of infusion parameters.

The ICM 202 may also include networking hardware for wirelessly connecting with a wireless hub or other network device 209 of network 10, 40, thereby providing network communications with server 30, PCU 12, or other network-enabled devices operably connected to a cloud-based system (e.g., via the network 10, 40). For example, patient information may be downloaded by the ICM 202 from an external system, limits of an infusion associated with the ICM set based on the patient information, and the flow rate of the infusion provided by a connected infusion device controlled by the ICM based on the limits and/or the patient information. In some implementations, the ICM 202 may be connected remotely to a PCU 12 or infusion pump through the server 30 and/or network 10, 40. For example, the ICM may be implemented as a mobile device or remote device 32.

In some implementations, the ICM 202 may be connected to multiple shared displays, and the displays may be used as display dashboards where multiple therapies are required to ease the screen real estate challenges in the critical care environment. A user interface may be provided to each display, with each interface including data modules (e.g., widgets) particular to the therapy associated with the display.

Additionally, in some implementations, where more than one ICM 202 is at use at a point of use, decision module redundancy can be used to support fault tolerant operation. For example, if I/O modules 205 are connected on an I/O bus, each may be accessed by other decision modules on the bus. Decision modules may operate in a redundant mode, taking over functions from compromised or failing members of the cluster.

In some implementations, the ICM 202 may include an integrated camera. In some implementations, the ICM 202 may incorporate facial tracking so that the display can pivot and rotate by a motorized bracket so that the display image is facing the HCP needing the information. This may be useful during surgery when the surgeon must move around the patient multiple times.

The camera may be incorporated as part of a vision system, which may further incorporate RFID or BLE used to track surgical instruments or devices being used at the bed side. With vision tracking, the camera may be used to identify the location of the ICM 202. This could be in addition to or in the alternative to tracking which currently requires an HCP to count and store the location of devices, instruments, bandages, and sponges to ensure that they are not misplaced or left in the patient during surgery, thus reducing errors (e.g., Retained Sponges and Instruments (RSI) errors). For example, the visual inspection system may further be integrated into the ICM to identify and count the number of sponges used, in order to better predict the blood volume included in them. This information can then be used by a hemodynamic control algorithm 216, to better manage the fluid volume needed to be infused to a patient. Significant loss of blood volume, together with patients having hemodynamic instability, has been associated with poor surgical outcomes. Accordingly, the hemodynamic and fluid management control system of the subject technology may provide improved safety and better patient recovery.

FIG. 2B depicts an example modular user interface 210 configured to dynamically connect to and receive data from different sensors to control different infusion therapies, according to aspects of the subject technology. User interface 210 is generated for display by ICM 202 and configured to facilitate connection of, based on user input, external sensors 204 with one or more medical devices 206, as described previously. The interface 210 provides a selection control 212, such as a menu, for selection of one or more therapies associated with an infusion device (or other medical device). The interface is also configured to display one or more graphic control modules 214 that receive sensor data, provide the data to a processing module, which in conjunction with the interface may control the infusion device. When a therapy 216 is selected, the interface 210 may dynamically reconfigure itself to display one or more predetermined modules 214 that correspond to the selected therapy.

Accordingly, the user interface 210 is configured to facilitate control of medical devices based on sensor data received from selected sensors. When a therapy is selected the available sensors (e.g., those connected to the ICM) may be displayed by the interface. A default user interface may be displayed, the user interface may be reconfigured based on which therapies, medical device(s), and/or sensors are selected for use. Data modules 214 (e.g., widgets) may be added or removed from the interface, as desired or required, to display patient and therapy data for the particular therapy being controlled by the ICM. Using the user interface 210 and/or data modules, a clinician may maintain control over an ongoing medical therapy (e.g., an infusion) according to the selected therapy type (e.g., anesthesia). The clinician may receive and respond to alerts, accept or reject recommendations, and set targets (e.g., drug concentrations).

According to various implementations, in operation, the ICM 202 operably connects to a medical device 206 and, upon recognizing the medical device, the user interface 210 software determines one or more predetermined therapies associated with the medical device. For example, the software may receive an identifier of the medical device upon connection to an infusion device and then query (e.g., over network 10) database 37 for the available therapies associated with the infusion device. The selection control 212 may then provide the therapies to the clinician for selection.

Each therapy may be associated with one or more modules 214 for controlling the infusion device based on input data. In some implementations, a module 214 may receive input data (e.g. parameters) from a clinician via the module while being displayed on the interface 210. In some implementations, each module may be associated with one or more external sensors 204, and at least a portion of the data may be received from the associated sensor(s) 204, when connected (e.g., to I/O modules 205). Sensors may be automatically discoverable and/or registrable by the system (e.g., using plug-and-play technology).

According to various implementations, the ICM 202 may host one or more clinical control algorithms 216. The algorithms 216 may be pushed down to the ICM over the communications network 10. Depending on therapy selection, I/O module 203 configuration the appropriate control algorithm can be chosen.

When a selection of a therapy is made, the system determines which sensor device(s) 204 associated with the therapy are operably connected to the ICM 202 and identifies and selects a corresponding algorithm(s) 216a configured to operate the connected medical device based on real-time data received from the connected sensor(s) 204. As used herein, “real-time” may refer to availability for processing at or near in time to the time the associated item is generated or detected. The algorithm(s) 216 may be stored locally in a memory of ICM 202 or, in some implementations, downloaded dynamically from a server 30 and/or database 37. In some implementations, an algorithm may include derived input values from monitoring or lab results and the like. Algorithms 216 may use advanced processing capabilities such as artificial intelligence, machine learning, and neural net processing. When an algorithm is stored locally in a memory of the ICM 202, the ICM 202 may verify the algorithm for use by the ICM 202. For example, the algorithm may be associated with an identifier or other information that can be used to determine properties of the algorithm such as version, distributor, author, etc. The identifier or other information may be assessed locally or using a server to determine whether the algorithm is suitable for use. For example, the algorithm stored may be out of date or recalled. In such instances, the ICM 202 may initiate download of a newer version. If the algorithm is verified and approved, the ICM 202 may continue as described.

According to various implementations, the ICM 202 identifies one or more modules 214a corresponding to the selected therapy and/or the selected algorithm(s) 216a, and generates a default organization of the identified module(s) 214a within the user interface 210. As depicted in FIG. 2B, the identified module(s) may then be displayed in the user interface 210 and begin receiving data from respective sensors devices 204a. Some modules 214 may be configured to receive user input via the user interface 210, such as configuration parameters associated with the operation of the infusion device. In some instances, a module 214a may prompt a user for the configuration parameter(s) and control, at least partially, the connected medical device based on the parameter(s) received from the user via the module(s).

When a new sensor is connected, the system may query the database 37 to determine what algorithm(s) is associated with the sensor, determine respective data module(s) 214, and update the user interface 210 to display the data module 214 associated with the new sensor. The interface 210 may then be used to control, in part via the determined data module, the medical device. Moreover, because of the modularity of the interface 210, when an updated algorithm becomes available for a particular sensor, the user interface 210 may be configured to load the new algorithm and replace a currently running algorithm with the newly loaded algorithm.

The user interface 210 may further include a data module 214 for displaying various checklists. Checklists may be used, for example, prior to a surgery to ensure familiarity with a patient's condition and the steps to be performed. This may performed as an ad hoc discussion between the caregivers involved. Infusion pump set-up is typically based on a user's experience and familiarity and may lead to errors, such as IV lines crossed between medications, incorrectly connected lines, improperly primed sets, IV fitments not properly wiped etc. The ICM 202, on detecting connection of a type of infusion device, may query the database 37 based on the type of infusion device and obtain a checklist for setup of the device. In some implementations, the checklist may be incorporated into a corresponding algorithm loaded by the ICM.

A checklist may include several steps requiring a confirmation from the clinician. The confirmation can be by way of a keypress on the ICM by the clinician, or by way of automatic activation by scanning a barcode, RFID, or connecting BLE enabled equipment. The checklist may define the order and confirmation of the steps needed to be performed. As an example, the ICM 202 may read information and communicate with the equipment to identify the IV extension set being used, the priming volume, which IV pump is being used for the medication, how the IV lines are connected, and/or setting occlusion alarm limits based on the IV setup.

The ICM 202 may further include alarm software. The fault, failure or attention needed at a medical device or sensor connected to the ICM 202 may generate one or more alarms. In cases where multiple devices are connected to the ICM, alarms that may not need immediate attention (e.g., a warning that the syringe is nearing empty), a hierarchy can be employed providing information on what needs to be addressed. The notification can be provided by means other than an audio alarm which would distract all clinicians involved in the surgery.

The ICM may also monitor all connected devices and use algorithms to predict potential faults prior to happening so that a clinician may act on them in a timely manner before a situation occurs requiring immediate action.

In some implementations, the user interface 210 may display a side bar providing alerts that a clinician can then interrogate to determine what actions need to be taken. The alerts can be color coded to indicate the source, status, and priority. As an example, yellow may indicate that items should be observed and provided direct attention when possible, such as pressure in the IV line is beginning to rise from baseline (e.g., a precursor to an occlusion), syringe volume down to a first predetermined threshold amount and/or not enough to complete an infusion order (e.g., 20% with X minutes until empty), the AC line became disconnected, or battery power provides X minutes of run time multiple sensor short term data interruptions (indicative of a loose connection).

Orange may indicate that items should be observed as soon as possible to ensure no interruption of treatment. For example, pressure in the IV line within a predetermined threshold range (e.g., at 80% of occlusion alarm), occlusion alarm is possible within a predetermined number of minutes, syringe volume down to a second predetermined threshold amount and/or not enough to complete an infusion (e.g., the infusion order is 10% with X minutes until empty), or the AC line became disconnected and/or battery power is at a predetermined level (e.g., at 10% and/or providing only X minutes of run time). Red may identify items that need immediate attention and/or that cause or have caused the system (pumps, ICM control) to stop requiring a clinician to manage and/or manually control the system. In such instances, the medical devices (e.g., pumps) may also provide an audio and/or visual alarm. A red alert may be provided, for example, when a syringe is empty, an IV line is occluded, the AC line is disconnected, or the battery power is depleted.

In some implementations, the software operating on the ICM 202 may combine information from the various sensors to identify a problem or a potential for a problem. For example, in the case of anesthesia, if a patient's bi-spectral index sensor 204 (BIS) is showing a rise in the BIS value together with changes in hemodynamic parameters, and a flow sensor 204 and/or sensor(s) within the pump 206a indicate that the rise in the BIS value is inversely proportional to the administration of drugs being infused, the ICM 202 may indicate that the patient is not responding as expected from receiving the medication. In such implementations, a hierarchy of potential faults can be displayed on the ICM 202 providing guidance to the clinician of what steps that need to be taken. For example, the fault color code on the ICM 202 may appear with an indication of one or more potential causes. The ICM 202 may also display a fault checklist showing potential checks to perform such as checking that the IV line is connected, not obstructed or the sensors are connected.

Computer program code for carrying out operations of the subject technology may be written in an object-oriented programming language such as, for example, JAVA®, Smalltalk, or C++. However, the computer program code for carrying out operations of the subject technology may also be written in conventional procedural programming languages, such as the “C” programming language, in an interpreted scripting language, such as Perl, or in a functional (or fourth generation) programming language such as Lisp, SML, Forth, or the like. The software may also be written to be compatible with HLA-7 requirements.

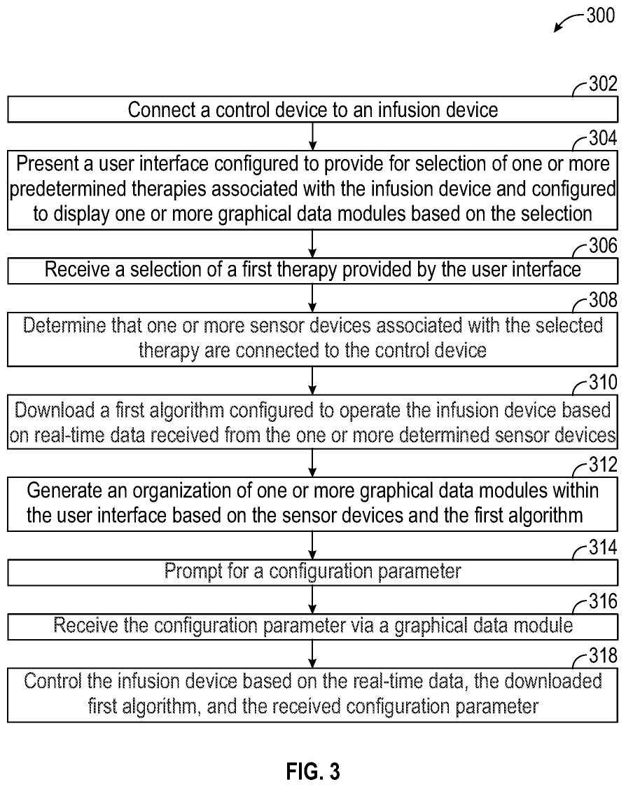

FIG. 3 depicts an example process for intelligently controlling an infusion, according to aspects of the subject technology. For explanatory purposes, the various blocks of example process 300 are described herein with reference to FIGS. 1 and 2, and the components and/or processes described herein. The one or more of the blocks of process 300 may be implemented by a control device such as, for example, by one or more computing devices including, for example, ICM 202, or component thereof. In some implementations, one or more of the blocks may be implemented apart from other blocks, and by one or more different processors or devices. Further for explanatory purposes, the blocks of example process 300 are described as occurring in serial, or linearly. However, multiple blocks of example process 300 may occur in parallel. In addition, the blocks of example process 300 need not be performed in the order shown and/or one or more of the blocks of example process 300 need not be performed.

In the depicted example, the ICM 202 operably connects to an infusion device 206a (302). For example, the ICM 202 may be operably connected to the infusion device by way of a wireless pairing or by way of a wired connection.

A user interface 210 is presented, for the control device (304). As described previously, the user interface may be configured to provide for selection one or more predetermined therapies associated with the infusion device and configured to display one or more graphical data modules based on the selection. In some implementations, the user interface 210 may be presented by the ICM 202. In some implementations, the user interface 210 may be generated by the ICM 202 and presented on an external display device (e.g., a display device associated with the infusion device or a mobile device). In some implementations, the user interface 210 may be generated by a server 30 and provided to and rendered by the ICM 202 or the external display device.

A selection of a first therapy of the one or more predetermined therapies is received at the control device (306). For example, with brief reference to FIG. 2B, therapies A-D may be displayed in a selection control 212 and a user/clinician may select a “Therapy C”. Based on receiving the selection of the first therapy (e.g., “Therapy C”), the ICM 202 may determine that one or more sensor devices for providing data associated with the selected first therapy are operably connected to the control device (308). Sensor devices 204 may include an infusion parameter sensor such as a flow rate monitor or pressure monitor, or may include a physiological monitor connected to a patient and configured to measure a physiological signal from the patient, such as an oxygen sensor, heart rate, or blood pressure sensor. Determining that the sensors are operably connected may include, for example, determining sensors 204 and/or sensor data associated with (or, e.g., required for) the selected therapy and then determining whether the sensors are connected to the ICM (e.g., by polling the I/O modules 205 or checking internal registration information).

In the depicted example, the ICM 202 obtains (e.g., downloads) a first algorithm to the ICM, from a server based on determining that the one or more sensor devices are operably connected to the control device (310). According to various implementations, the first algorithm is configured to operate the infusion device based on real-time data received from the one or more sensor devices. In some implementations, the first algorithm is configured to operate the infusion device in a closed-loop mode based on the real-time data received from the one or more sensor devices. In some implementations, the ICM 202 confirms that the one or more sensor devices are associated with the first therapy and a type of the infusion device before downloading the first algorithm and proceeding to step 312 (below).

The ICM 202 then generates an organization of one or more respective graphical data modules within the user interface based on the one or more sensor devices and the downloaded first algorithm (312). In this regard, at least one of the one or more respective graphical data modules may be configured to receive a configuration parameter associated with operation of the infusion device.

As previously described with regard to FIG. 2B, before the organization of the one or more respective graphical data modules is generated, the ICM 202 may select the one or more respective graphical data modules 214a from a plurality of predetermined graphical data modules 214 based on the selection of the first therapy and a type of the one or more sensor devices 204a.

With further reference to FIG. 3, the ICM 202 prompts for a configuration parameter (314), and receives, via the at least one respective graphical data module, the configuration parameter (316). In some implementations, the prompting may be by way of the respective graphical data module providing a designated control for receiving input of the parameter. In some implementations, the parameter may be required for operation and the module may specifically request input of the parameter before the ICM 202 initiates control of an infusion.

The ICM 202 then controls the infusion device based on the real-time data received from the one or more sensor devices, the downloaded first algorithm, and the received configuration parameter (318). In some implementations, where the first algorithm facilitates a closed-loop mode, the infusion device may be controlled in a closed-loop based on the real-time data received from the one or more sensor devices, the downloaded first algorithm, and the received configuration parameter.

The ICM 202 may act as an extension of the PCU 12 or infusion pump via user interface 210. For example, the ICM 202 may allow for input (e.g., via a module 214a) of an infusion parameter such as a flow rate or an amount of a medication provided by the infusion device, and control the infusion pump based on the input. Moreover, the ICM 202 may download data from other sources, make determinations based on the obtained data and user input, and control medical devices based on analysis of that data.

In some implementations, the ICM 202 may receive information from the infusion device which pertains to or identifies a patient. The ICM 202 may connect to a cloud-based system configured to accumulate patient data (e.g., via a network) and download patient order information pertaining to the patient from the cloud-based system. The ICM 202 may then determine limits on the infusion information based on the patient order information and control the flow rate of the medication provided by the infusion device based on the determined limits. The limits may be determined, for example, by way of indexing a lookup table based on a patient identification or querying the server based on the identification.

According to various implementations, the modularity of the system provides for extensibility and a longer lifecycle through dynamic updates. For example, a new sensor may be operably connected to the ICM 202. Upon the ICM 202 detecting the new sensor, the ICM 202 may download a second algorithm associated with the new sensor and update the user interface to display a new data module associated with the new sensor. Data may then be received from the new sensor and displayed via the new data module. The infusion device may then be controlled within the closed-loop mode based on real-time data received from the new sensor and the one or more sensor devices, the downloaded first and second algorithms, and the received configuration parameter.

The system may later receive a new algorithm associated with a first sensor of the one or more sensor devices. The new algorithm may be configured to operate the infusion device based on real-time data received from the first sensor. The ICM 202 may automatically replace the first algorithm with the third algorithm and begin controlling the infusion device based on real-time data received from the one or more sensor devices, the new algorithm and the received configuration parameter.

During operation, a sensor measurement value is received from a respective sensor and provided to the first algorithm. The algorithm processes the value and, based at least in part on that processing, provides a parameter for adjusting an operation of the infusion device. The ICM 202 may then adjust the operation of the infusion device based on the received parameter.

For the case of multiple simultaneous closed-loop therapies (e.g., IV Glycemic control and Hemodynamic stability), the ICM 202 could host simultaneous therapies running one or more closed-loop algorithms with simultaneous connection to the different sensors and actuators. An alternate implementation could use multiple ICMs each running a single closed-loop therapy.

In some implementations, multiple data modules may utilize the same sensor(s) and/or the same algorithm(s). In some implementations, the ICM 202 (e.g., via the user interface 210 and/or the modules therein) may control multiple (different) medical devices at the same time based on data from the same sensor(s). In one example, ICM 202 may be configured to, upon connecting to a second medical device and receiving a second selection of a second therapy (e.g., from the selection control 212), confirm that at least one corresponding sensor(s) is operably connected to the ICM 202, identify and obtain (e.g., download) the corresponding algorithm(s), and display at least one graphical data module associated with receiving data from the at least one sensor(s) and with controlling the medical device. The ICM 202 may then control the second medical device based on the real-time data received from the at least one sensor and the second algorithm while simultaneously controlling the infusion device.

In some implementations, multiple data modules 214 may utilize data from the same sensor(s). For example, at least one sensor 204 corresponding to the first selected therapy may be one of the one or more sensor devices for providing data associated with the second selected therapy. In this manner, an infusion device and a different medical device may then be controlled based on same data received from the same sensor 204. Because each device may be controlled by a different algorithm (and/or, e.g., data module 214), the infusion device may be, for example, controlled based on input of the same data to the first algorithm and the medical device may be controlled based on input of the same data to the second algorithm.

In some implementations, the ICM 202 may be operably connected to two infusion devices. In this regard, a second algorithm may be downloaded to the ICM 202 from the server, and the second infusion device controlled based on the real-time data received from the one or more sensor devices and the second algorithm. The second infusion device may be operated based on an additional sensor connected to the control device. Based on detecting the additional sensor being detected (e.g., by the ICM 202), the second algorithm may be downloaded from the server, and the user interface updated to display an additional data module associated with the additional sensor, as described previously. Data may be received from the additional sensor and displayed the received data via the additional data module. And, in some implementations, the second infusion device may be controlled in the closed-loop mode based on real-time data received from the additional sensor, the downloaded first and/or second algorithms, and/or the received configuration parameter.

Many of the above-described example 300, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Applications of the ICM to control infusion pumps and/or other medical devices, according to various aspects of the subject technology, may be used for closed-loop treatment of a variety of conditions such as anesthesia, blood conditions, blood transfusions, care or medicinal transitions, chemotherapy, enteral therapy, exfiltration, fluid balance conditions, glycemic conditions, hemodynamic conditions, hydration, infiltration, nutritional care, patient controlled analgesic, patient or fluid temperature condition, vasopressor ventilation,. In such implementations, the ICM 202 may receive input based on one or more intelligent models and/or protocols and/or simulations to make decisions for controlling an infusion device. Such models, protocols, and simulations may be based, at least in part, on machine learning algorithms that process training data input based on a population of patients having similar conditions to the patient being treated. For example, a vasopressor based therapy can require frequent boluses, and adjustment of infusion rates expediently to avoid harmful periods of hypotension or hypertension. The foregoing implementation may also be used to control sepsis treatment which requires antibiotic administration and fluid resuscitation to correct hypotension. Such IV fluid management is important for sepsis patients and controlling the timing, type, and amount of fluid administered is critical since excess quantities of IV fluids could also be detrimental.

As another example, anesthesia may require a predetermined amount of an administration of drugs to achieve the required end points of hypnosis, immobility, and suppression of reflexes during surgery. It may be given as the combination of a hypnotic and an opioid, with the anesthesiologist manually titrating doses or infusion rates of the 2 drugs to provide the best balance. Using a bispectral index sensor 204 (BIS), the ICM 202 may monitor the depth of anesthesia and remotely control the infusion device to administer a proper amount of an IV drug(s), while preventing awareness or excessive anesthetic depth during medical treatment, thereby improving patients' outcomes.

As another example, the ICM 202 may be connected to a sensor 204 configured as a blood glucose monitor and, based on intelligent modeling and continuous blood glucose measurements, maintain a patient's blood glucose by way of controlling an infusion device's administration of insulin and/or dextrose solution(s). Blood glucose (BG) disorders, such as stress-induced hypoglycemia and hyperglycemia, can be common complications in patients in the ICU. In addition, patients with type 1 and 2 diabetes may be susceptible to hyperglycemia, as well as severe hypoglycemia as a result of overcorrection with insulin. For this reason, IV infusions of insulin that are controlled by the ICM 202 using a closed-loop configuration with inputs from continuous BG measurements is an ideal application for IV infusions of inulin and dextrose to maintain BG levels within the desired range.

FIG. 4 is a conceptual diagram illustrating an example electronic system 400 for intelligently controlling an infusion, according to aspects of the subject technology. Electronic system 400 may be a computing device for execution of software associated with one or more portions or steps of process 400, or components and processes provided by FIGS. 1-3, including but not limited to information system server 30, database 37, computing hardware within patient care device 12, control unit 14, a respective module 16, 18, 20, 22, or a remote device 32 (e.g., a mobile device). Electronic system 400 may be representative, in combination with the disclosure regarding FIGS. 1-4. In this regard, electronic system 400 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 414, an output device interface 406, and one or more network interfaces 416. In some implementations, electronic system 400 may include or be integrated with other computing devices or circuitry for operation of the various components and processes previously described.

Bus 408 collectively represents all system, peripheral, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process, in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such as a random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves instructions to execute and data to process in order to execute the processes of some implementations.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”). Output device interfaces 406 enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 4, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 400 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

The subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

### ILLUSTRATION OF SUBJECT TECHNOLOGY AS CLAUSES

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. An infusion control device, comprising: a non-transitory machine-readable memory; and a processor operably connected to a display and the memory, the processor configured to: provide a user interface to the display; prompt, via the user interface, a user to select a therapy; receive a selected therapy from the user interface; confirm that one or more sensor devices required for the selected therapy are operably connected to the infusion control device; confirm that one or more intravenous infusion devices are operably connected to the infusion control device and that the one or more intravenous infusion devices are controllable based on information derivable from the sensor devices; obtain a first algorithm configured to operate the one or more intravenous infusion devices based on real-time data received from the one or more sensor devices; and facilitate performance of the selected therapy based on sensor data received from the one or more sensor devices, wherein at least a portion of the sensor data is processed via the first algorithm.

Clause 2. The infusion control device of Clause 1, wherein the processor is further configured to: operably connect the infusion control device to a first infusion device of the one or more intravenous infusion devices, the infusion control device being physically distinct from the one or more intravenous infusion devices; and based on receiving the selection of the therapy: generate a default organization of one or more respective graphical data modules within the user interface based on the one or more sensor devices and the first algorithm, wherein at least one of the one or more respective graphical data modules is configured to receive a configuration parameter associated with operation of the first infusion device; receive, via the at least one respective graphical data module, the configuration parameter; and control the first infusion device based on the real-time data received from the one or more sensor devices, the first algorithm, and the received configuration parameter.

Clause 3. The infusion control device of Clause 2, wherein the first algorithm is configured to operate the first infusion device in a closed-loop mode based on the real-time data received from the one or more sensor devices, wherein controlling the first infusion device comprises: controlling the first infusion device in the closed-loop mode based on the real-time data received from the one or more sensor devices, the downloaded first algorithm, and the received configuration parameter.

Clause 4. The infusion control device of Clause 3, wherein the processor is further configured to: detect an additional sensor operably connected to the control device; based on detecting the additional sensor: download to the control device, from a server, a second algorithm associated with the additional sensor; update the user interface to display a additional data module associated with the additional sensor; receive data from the additional sensor and displaying the received data via the additional data module; and control the first infusion device in the closed-loop mode based on real-time data received from the additional sensor and the one or more sensor devices, the downloaded first and second algorithms, and the received configuration parameter.

Clause 5. The infusion control device of any one of Clauses 2 through 4, wherein the processor is further configured to: receive a third algorithm associated with a first sensor of the one or more sensor devices, the third algorithm configured to operate the first infusion device based on real-time data received from the first sensor; automatically replace the first algorithm with the third algorithm; and control the first infusion device based on real-time data received from the one or more sensor devices, the third algorithm and the received configuration parameter.

Clause 6. The infusion control device of any one of Clauses 2 through 5, wherein the one or more sensor devices comprises a physiological monitor connected to a patient and configured to measure a physiological signal from the patient, and wherein the real-time data includes a measurement value of the physiological signal, wherein the processor is further configured to: provide the measurement value to the first algorithm; receive a parameter for adjusting an operation of the first infusion device from the first algorithm based on providing the measurement value to the first algorithm; and adjust the operation of the first infusion device based on the received parameter.

Clause 7. The infusion control device of any one of Clauses 2 through 6, wherein the processor is further configured to, before generating the default organization of the one or more respective graphical data modules: select the one or more respective graphical data modules from a plurality of predetermined graphical data modules based on the selection of the selected therapy and a type of the one or more sensor devices.

Clause 8. The infusion control device of any one of Clauses 2 through 7, wherein the processor is further configured to: operably connect the control device to a medical device; receive a second selection of a second therapy from the user interface; and based on receiving the second selection: confirm that at least one sensor corresponding to the second therapy is operably connected to the control device; download, from a server, a second algorithm configured to operate the medical device based on real-time data received from the at least one sensor; display at least one graphical data module associated with receiving data from the at least one sensor and with controlling the medical device; and control the medical device based on the real-time data received from the at least one sensor and the second algorithm while simultaneously controlling the first infusion device.

Clause 9. The infusion control device of any one of Clauses 2 through 8, wherein the processor is further configured to: confirm that the one or more sensor devices are associated with the selected therapy and a type of the first infusion device before downloading the first algorithm and generating the organization within the user interface.

Clause 10. The infusion control device of any one of Clauses 2 through 9, wherein the processor is further configured to: operably connect the infusion control device to a second infusion device of the one or more intravenous infusion devices, the infusion control device being physically distinct from the second infusion device; download to the control device, from a server, a second algorithm; and control the second infusion device based on the real-time data received from the one or more sensor devices and the second algorithm.

Clause 11. The infusion control device of Clause 10, wherein the processor is further configured to: adjust a parameter of the second infusion device based on an alert from the first infusion device.

Clause 12. The infusion control device of any one of Clauses 1 through 11, further comprising the display.

Clause 13. A method of intelligently controlling an intravenous infusion, comprising: receiving an indication that a control device is operably connected to one or more intravenous infusion devices, the control device being physically distinct from the one or more intravenous infusion devices; presenting, for the control device, a user interface configured to provide for selection one or more predetermined therapies associated with the one or more intravenous infusion devices and configured to display one or more graphical data modules based on the selection; receiving, at the control device, a selection of a first therapy of the one or more predetermined therapies provided by the user interface; and based on receiving the selection of the first therapy, at the control device: determining that one or more sensor devices for providing data associated with the selected first therapy are operably connected to the control device; downloading to the control device, from a server based on determining that the one or more sensor devices are operably connected to the control device, a first algorithm configured to operate the one or more intravenous infusion devices based on real-time data received from the one or more sensor devices; generating a default organization of one or more respective graphical data modules within the user interface based on the one or more sensor devices and the downloaded first algorithm, wherein at least one of the one or more respective graphical data modules is configured to receive a configuration parameter associated with operation of the one or more intravenous infusion devices; prompting for the configuration parameter; receiving, via the at least one respective graphical data module, the configuration parameter; and controlling the one or more intravenous infusion devices based on the real-time data received from the one or more sensor devices, the downloaded first algorithm, and the received configuration parameter.

Clause 14. The method of Clause 13, wherein the first algorithm is configured to operate a first infusion device in a closed-loop mode based on the real-time data received from the one or more sensor devices, wherein controlling the first infusion device comprises: controlling the first infusion device in the closed-loop mode based on the real-time data received from the one or more sensor devices, the downloaded first algorithm, and the received configuration parameter.

Clause 15. The method of Clause 14, further comprising: detecting a new sensor operably connected to the control device; based on detecting the new sensor: downloading to the control device, from the server, a second algorithm associated with the new sensor; updating the user interface to display a new data module associated with the new sensor; receiving data from the new sensor and displaying the received data via the new data module; and controlling the first infusion device in the closed-loop mode based on real-time data received from the new sensor and the one or more sensor devices, the downloaded first and second algorithms, and the received configuration parameter.

Clause 16. The method of any one of Clauses 13 through 15, further comprising: confirming that the one or more sensor devices are associated with the first therapy and a type of a first infusion device before downloading the first algorithm and generating the default organization within the user interface.

Clause 17. The method of any one of Clauses 13 through 16, further comprising: receiving a third algorithm associated with a first sensor of the one or more sensor devices, the third algorithm configured to operate the first infusion device based on real-time data received from the first sensor; automatically replacing the first algorithm with the third algorithm; and controlling the first infusion device based on real-time data received from the one or more sensor devices, the third algorithm and the received configuration parameter.

Clause 18. The method of any one of Clauses 13 through 17, wherein the one or more sensor devices comprises a physiological monitor connected to a patient and configured to measure a physiological signal from the patient, and wherein the real-time data includes a measurement value of the physiological signal, the method further comprising: providing the measurement value to the first algorithm; receiving a parameter for adjusting an operation of the first infusion device from the first algorithm based on providing the measurement value to the first algorithm; and adjusting the operation of the first infusion device based on the received parameter.

Clause 19. The method of any one of Clauses 13 through 18, further comprising, before generating the default organization of the one or more respective graphical data modules: selecting the one or more respective graphical data modules from a plurality of predetermined graphical data modules based on the selection of the first therapy and a type of the one or more sensor devices.

Clause 20. The method of any one of Clauses 13 through 19, further comprising: operably connecting the control device to a medical device; receiving, for the medical device from the user interface, a second selection of a second therapy of the one or more predetermined therapies provided by the user interface; and based on receiving the second selection: confirming that at least one sensor corresponding to the second therapy is operably connected to the control device; downloading, from the server, a second algorithm configured to operate the medical device based on real-time data received from the at least one sensor; displaying at least one graphical data module associated with receiving data from the at least one sensor and with controlling the medical device; and controlling the medical device based on the real-time data received from the at least one sensor and the second algorithm while simultaneously controlling the first infusion device.

Clause 21. The method of Clause 20, wherein the at least one sensor corresponding to the second therapy is one of the one or more sensor devices for providing data associated with the selected first therapy, the method further comprising: controlling the first infusion device and the medical device based on same data received from the at least one sensor, the first infusion device being controlled based on input of the same data to the first algorithm and the medical device being controlled based on input of the same data to the second algorithm.

Clause 22. The method of any one of Clauses 13 through 21, further comprising: identifying, based on information received from the first infusion device, a patient designated to receive therapy from the first infusion device; operably connecting the control device to a cloud-based system configured to accumulate patient data; downloading patient order information pertaining to the patient from the cloud-based system; and receiving, at the control device from the first infusion device, infusion information comprising a flow rate or an amount of a medication provided by the first infusion device; determining, at the control device, limits on the infusion information based on the patient order information; and controlling, at the control device, the flow rate of the medication provided by the first infusion device based on the determined limits on the infusion information.

Clause 23. The method of any one of Clauses 13 through 22, further comprising: operably connecting the infusion control device to a second infusion device of the one or more intravenous infusion devices, the infusion control device being physically distinct from the second infusion device; downloading to the control device, from a server, a second algorithm; and controlling the second infusion device based on the real-time data received from the one or more sensor devices and the second algorithm.

Clause 24. The method of Claim 23, further comprising: adjusting a parameter of the second infusion device based on an alert from the first infusion device.

Clause 25. A non-transitory machine-readable medium storing instructions thereon that, when executed by a processor, cause the processor to perform a method according to any one of Clauses 13 through 24.

### FURTHER CONSIDERATION

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

1. An infusion control device, comprising:
a non-transitory machine-readable memory; and
a processor operably connected to a display and the memory, the processor configured to:
provide a user interface to the display;
prompt, via the user interface, a user to select a therapy;
receive a selected therapy from the user interface;
based on receiving the selected therapy:
confirm that one or more sensor devices required for the selected therapy are operably connected to the infusion control device;
confirm that one or more intravenous infusion devices are operably connected to the infusion control device and that the one or more intravenous infusion devices are controllable based on information derivable from the sensor devices;
download a first algorithm, the first algorithm configured to operate the one or more intravenous infusion devices based on real-time sensor data received from the one or more sensor devices;
select one or more graphical data modules particular to the selected therapy for display within the user interface based on a type of the one or more sensor devices and the selected therapy, wherein each selected graphical data module is associated with and configured to receive sensor data from a respective sensor device of the sensor devices and provide the sensor data to a processing module, wherein at least one of the respective graphical data modules is configured to receive a configuration parameter associated with operation of a first infusion device of the one or more intravenous infusion devices; and
dynamically reconfigure the user interface to display, within the user interface, a default organization of the selected one or more graphical data modules based on the first algorithm;
operably connect the infusion control device to the first infusion device, the infusion control device being physically distinct from the one or more intravenous infusion devices;
receive, via the at least one respective graphical data module, the configuration parameter; and
control the first infusion device based on the sensor data received by the processing module from the selected one or more graphical data modules, the first algorithm, and the received configuration parameter.

2. The infusion control device of claim 1, wherein the first algorithm is configured to operate the first infusion device in a closed-loop mode based on the real-time data received from the one or more sensor devices, wherein controlling the first infusion device comprises:
controlling the first infusion device in the closed-loop mode based on the real-time data received from the one or more sensor devices, the downloaded first algorithm, and the received configuration parameter.

3. The infusion control device of claim 2, wherein the processor is further configured to:
detect an additional sensor operably connected to the control device;
based on detecting the additional sensor:
download to the control device, from a server, a second algorithm associated with the additional sensor;
update the user interface to display an additional data module associated with the additional sensor;
receive data from the additional sensor and displaying the received data via the additional data module; and
control the first infusion device in the closed-loop mode based on real-time data received from the additional sensor and the one or more sensor devices, the downloaded first and second algorithms, and the received configuration parameter.

4. The infusion control device of claim 1, wherein the processor is further configured to:
receive a third algorithm associated with a first sensor of the one or more sensor devices, the third algorithm configured to operate the first infusion device based on real-time data received from the first sensor;
automatically replace the first algorithm with the third algorithm; and
control the first infusion device based on real-time data received from the one or more sensor devices, the third algorithm and the received configuration parameter.

5. The infusion control device of claim 1, wherein the one or more sensor devices comprises a physiological monitor connected to a patient and configured to measure a physiological signal from the patient, and wherein the real-time data includes a measurement value of the physiological signal, wherein the processor is further configured to:
provide the measurement value to the first algorithm;
receive a parameter for adjusting an operation of the first infusion device from the first algorithm based on providing the measurement value to the first algorithm; and
adjust the operation of the first infusion device based on the received parameter.

6. The infusion control device of claim 1, wherein the processor is further configured to, before generating the default organization of the one or more respective graphical data modules:
select the one or more respective graphical data modules from a plurality of predetermined graphical data modules based on the selection of the selected therapy and a type of the one or more sensor devices.

7. The infusion control device of claim 1, wherein the processor is further configured to:
operably connect the control device to a medical device;
receive a second selection of a second therapy from the user interface; and
based on receiving the second selection:
confirm that at least one sensor corresponding to the second therapy is operably connected to the control device;
download, from a server, a second algorithm configured to operate the medical device based on real-time data received from the at least one sensor;
display at least one graphical data module associated with receiving data from the at least one sensor and with controlling the medical device; and
control the medical device based on the real-time data received from the at least one sensor and the second algorithm while simultaneously controlling the first infusion device.

8. The infusion control device of claim 1, wherein the processor is further configured to:
confirm that the one or more sensor devices are associated with the selected therapy and a type of the first infusion device before downloading the first algorithm and generating the organization within the user interface.

9. The infusion control device of claim 1, wherein the processor is further configured to:
operably connect the infusion control device to a second infusion device of the one or more intravenous infusion devices, the infusion control device being physically distinct from the second infusion device;
download to the control device, from a server, a second algorithm; and
control the second infusion device based on the real-time data received from the one or more sensor devices and the second algorithm.

10. The infusion control device of claim 9, wherein the processor is further configured to:
adjust a parameter of the second infusion device based on an alert from the first infusion device.

11. The infusion control device of claim 1, further comprising the display.

12. A method of intelligently controlling an intravenous infusion, comprising:
receiving an indication that a control device is operably connected to one or more intravenous infusion devices, the control device being physically distinct from the one or more intravenous infusion devices;
presenting, for the control device, a user interface configured to provide for selection one or more predetermined therapies associated with the one or more intravenous infusion devices and configured to display one or more graphical data modules based on the selection;
receiving, at the control device, a selection of a first therapy of the one or more predetermined therapies provided by the user interface; and
based on receiving the selection of the first therapy, at the control device:
determining that one or more sensor devices for providing data associated with the selected first therapy are operably connected to the control device;
downloading to the control device, from a server based on determining that the one or more sensor devices are operably connected to the control device, a first algorithm configured to operate the one or more intravenous infusion devices based on real-time data received from the one or more sensor devices;
select one or more respective graphical data modules particular to the selected first therapy within the user interface based on a type of the one or more sensor devices and the selected therapy, wherein each selected graphical data module is associated with and configured to receive sensor data from a respective sensor device of the sensor devices and provide the sensor data to a processing module, and at least one of the one or more respective graphical data modules is configured to receive a user input of a configuration parameter associated with operation of the one or more intravenous infusion devices; and
dynamically reconfiguring the user interface to display, within the user interface, a default organization of the selected one or more graphical data modules based on the first algorithm;
prompting for the configuration parameter;
receiving, via the at least one respective graphical data module, the configuration parameter; and
controlling the one or more intravenous infusion devices based on the real-time data received by the processing module from the one or graphical data modules, the downloaded first algorithm, and the received configuration parameter.

13. The method of claim 12, wherein the first algorithm is configured to operate a first infusion device in a closed-loop mode based on the real-time data received from the one or more sensor devices, wherein controlling the first infusion device comprises:
controlling the first infusion device in the closed-loop mode based on the real-time data received from the one or more sensor devices, the downloaded first algorithm, and the received configuration parameter.

14. The method of claim 13, further comprising:
detecting a new sensor operably connected to the control device;
based on detecting the new sensor:
downloading to the control device, from the server, a second algorithm associated with the new sensor;
updating the user interface to display a new data module associated with the new sensor;
receiving data from the new sensor and displaying the received data via the new data module; and
controlling the first infusion device in the closed-loop mode based on real-time data received from the new sensor and the one or more sensor devices, the downloaded first and second algorithms, and the received configuration parameter.

15. The method of claim 12, further comprising:
confirming that the one or more sensor devices are associated with the first therapy and a type of a first infusion device before downloading the first algorithm and generating the default organization within the user interface.

16. The method of claim 12, further comprising:
receiving a third algorithm associated with a first sensor of the one or more sensor devices, the third algorithm configured to operate the first infusion device based on real-time data received from the first sensor;
automatically replacing the first algorithm with the third algorithm; and
controlling the first infusion device based on real-time data received from the one or more sensor devices, the third algorithm and the received configuration parameter.

17. The method of claim 12, wherein the one or more sensor devices comprises a physiological monitor connected to a patient and configured to measure a physiological signal from the patient, and wherein the real-time data includes a measurement value of the physiological signal, the method further comprising:
providing the measurement value to the first algorithm;
receiving a parameter for adjusting an operation of the first infusion device from the first algorithm based on providing the measurement value to the first algorithm; and
adjusting the operation of the first infusion device based on the received parameter.

18. The method of claim 12, further comprising, before generating the default organization of the one or more respective graphical data modules:
selecting the one or more respective graphical data modules from a plurality of predetermined graphical data modules based on the selection of the first therapy and a type of the one or more sensor devices.

19. The method of claim 12, further comprising:
operably connecting the control device to a medical device;
receiving, for the medical device from the user interface, a second selection of a second therapy of the one or more predetermined therapies provided by the user interface; and
based on receiving the second selection:
confirming that at least one sensor corresponding to the second therapy is operably connected to the control device;
downloading, from the server, a second algorithm configured to operate the medical device based on real-time data received from the at least one sensor;
displaying at least one graphical data module associated with receiving data from the at least one sensor and with controlling the medical device; and
controlling the medical device based on the real-time data received from the at least one sensor and the second algorithm while simultaneously controlling the first infusion device.

20. The method of claim 19, wherein the at least one sensor corresponding to the second therapy is one of the one or more sensor devices for providing data associated with the selected first therapy, the method further comprising:
controlling the first infusion device and the medical device based on same data received from the at least one sensor, the first infusion device being controlled based on input of the same data to the first algorithm and the medical device being controlled based on input of the same data to the second algorithm.

21. The method of claim 12, further comprising:
identifying, based on information received from the first infusion device, a patient designated to receive therapy from the first infusion device;
operably connecting the control device to a cloud-based system configured to accumulate patient data;
downloading patient order information pertaining to the patient from the cloud-based system; and
receiving, at the control device from the first infusion device, infusion information comprising a flow rate or an amount of a medication provided by the first infusion device;
determining, at the control device, limits on the infusion information based on the patient order information; and
controlling, at the control device, the flow rate of the medication provided by the first infusion device based on the determined limits on the infusion information.

22. The method of claim 12, further comprising:
operably connecting the infusion control device to a second infusion device of the one or more intravenous infusion devices, the infusion control device being physically distinct from the second infusion device;
downloading to the control device, from a server, a second algorithm; and
controlling the second infusion device based on the real-time data received from the one or more sensor devices and the second algorithm.

23. The method of claim 22, further comprising:
adjusting a parameter of the second infusion device based on an alert from the first infusion device.

24. A non-transitory machine-readable medium storing instructions thereon that, when executed by a processor, cause the processor to perform a method according to claim 12.
